(19) United States
(12) Patent Application Publication
(10) Pub. No.: US 2008/0119444 A1
(43) Pub. Date: May 22, 2008

(54) Methods and compositions for the treatment of cancer

(76) Inventor: Steven Lehrer, New York, NY (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/600,572

(22) Filed: Nov. 16, 2006

(51) Int. Cl. A61K 31/566; A61P 35/00; A61K 31/4015; A61K 31/351; A61K 31/21; A61K 31/4965; A61K 31/47; A61K 31/404; A61K 31/4196

(52) U.S. Cl. 514/171; 514/383; 514/423; 514/415; 514/460; 514/311; 514/529; 514/275

(57) ABSTRACT

The present invention provides methods to treat neoplastic disorders in a human comprising the use of aromatase inhibitors and HMG-CoA reductase inhibitors (statins).

### FIELD OF THE INVENTION

This invention relates to the field of oncology. More specifically, the present invention relates to compositions and methods for the treatment of neoplastic disorders, particularly estrogen dependent cancers, by administering at least one aromatase inhibitor and at least one HMG-CoA reductase inhibitor (statin).

### BACKGROUND OF THE INVENTION

Several publications and patent documents are cited throughout the specification in order to describe the state of the art to which this invention pertains. Each of these citations is incorporated herein by reference as though set forth in full.

In 2005, an estimated 211,240 new cases of female breast cancer were diagnosed in the United States. The overall incidence rate of breast cancer in US women is 132.9 per 100,000 and this rate rises with age. For women ages 60 to 64 years, the incidence is 394.7 per 100,000. Two thirds of newly diagnosed breast cancers in women older than 50 years are localized to the breast (Come, S. E. (2006) JAMA, 295:1434-42).

The prognosis for stage I breast cancer is variable, with recurrence-free survival ranging from 70% to greater than 90%. Tumor size, histological grade, proliferative index, and expression of hormone receptors are the most important factors in predicting outcome and in selecting therapy. Invasive tumors smaller than 1 cm, histological grade 1, low proliferative index, and positive for both estrogen (ER+) and progesterone (PR+) receptors represent the most favorable prognosis; conversely, larger tumor size, higher grade and/or proliferative index, and lack of hormone receptor expression are adverse features. The presence of lymphovascular invasion in the tumor specimen, overexpression or amplification of Her-2/neu, and very young patient age (<35 years) are additional unfavorable factors (Come, S. E. (2006) JAMA, 295:1434-42).

For stage I breast cancer, primary treatment consists of either mastectomy or the combination of lumpectomy (excision of the tumor with free margins) and radiation therapy to the breast. These alternatives provide equivalent long-term disease-free, distant disease-free, and overall survival. Lumpectomy without radiation therapy is associated with a higher risk of recurrence in the involved breast. A meta-analysis of 6 trials with greater than 10 years of follow-up in which a total of 4,177 women were randomized to undergo breast-conserving surgery with or without radiation found a 68% relative reduction in local recurrence for patients who received radiation (7%) compared to those who did not (22%, P<0.001). There is some evidence that the risk of local tumor failure after conservative surgery alone in women 65 years of age and older with small, low-grade, node-negative tumors is low enough that radiation therapy may be omitted in this specific subset (Come, S. E. (2006) JAMA, 295:1434-42).

In many women with early breast cancer, medical therapy is added to primary treatment. Systemic treatment with either chemotherapy, tamoxifen, or both has been shown to decrease local recurrence rates in the breast in patients undergoing conservative surgery (lumpectomy) and radiation therapy. However, a decision as to whether, and if so which, adjuvant systemic therapy is added to primary therapy depends principally on an assessment of the risk of subsequent distant tumor recurrence. Furthermore, the impact of these systemic therapies on local control in patients undergoing conservative surgery without radiation therapy is not well studied, and they are not generally considered an alternative to radiation therapy in patients desiring breast conservation (Come, S. E. (2006) JAMA, 295:1434-42).

Data from numerous studies and from a series of meta-analyses performed every 5 years since 1985 demonstrate that, when added to primary therapy, both endocrine therapy and chemotherapy can reduce recurrence and mortality in women with early breast cancer when added to primary therapy. The benefit of endocrine therapy is limited to patients with tumors that express hormone receptors. Among postmenopausal women, 79% have tumors that are ER+ and 53% are PR+. The majority of the latter are also ER+ as fewer than 5% of tumors are ER−PR+. Expression of PR is an independent predictor of favorable outcome on endocrine therapy. Clearly, a need exists for the development of new treatment strategies to prevent the growth and spread of breast cancer.

### SUMMARY OF THE INVENTION

In accordance with the present invention, anti-cancer compositions are provided which comprise at least one aromatase inhibitor and at least one HMG-CoA reductase inhibitor (statin) or a pharmaceutically acceptable salt thereof or a solvate thereof in a therapeutic combination.

Aromatase inhibitors include, but are not limited to anastrozole, letrozole, or exemestane. The HMG-CoA reductase inhibitors (statins) include but are not limited to atorvastatin (e.g., LIPITOR®), fluvastatin (e.g., LESCOL®, CANEF®), lovastatin (e.g., MEVACOR®), mevastatin, pitavastatin, pravastatin (e.g., PRAVACHOL® or SELEKTINE®), rosuvastatin (e.g., CRESTOR®), and simvastatin (e.g., ZOCOR®). In a preferred embodiment, the HMG-CoA reductase inhibitor and the aromatase inhibitor are present in the composition at therapeutically effective amounts and may be incorporated into a single pill, tablet, or capsule.

In accordance with another aspect of the instant invention, methods are provided for the treatment of cancer comprising the administration of the pharmaceutical compositions described herein alone or in combination with other agents conventionally used in the treatment of breast cancer.

### DETAILED DESCRIPTION OF THE INVENTION

### Tamoxifen

The selective estrogen receptor modulator (SERM) tamoxifen has been the standard of care as adjuvant endocrine therapy for more than 20 years. A recently published meta analysis, initiated in 2000, reports data from 71 trials of adjuvant tamoxifen involving more than 80,000 women. Fifteen-year observations on the effects of treatment versus control and of the risks of therapy are now available. These studies found that 5 years of tamoxifen treatment reduced the annual rates of recurrence by 41% and mortality by 34% in women with ER+disease (Come, S. E. (2006) JAMA, 295:1434-42).

The reduction in rate of recurrence is maintained during tamoxifen treatment and for about 5 years after cessation of tamoxifen treatment. Beyond year 10, the recurrence rates in the treatment and control groups are similar, but the gains of the first 10 years are maintained. In addition, the reduction in mortality rate for women treated with tamoxifen compared with controls is still evident at year 15. At 15 years, there is an absolute reduction in recurrence of 11.8% (P<0.001) and in mortality of 9.2% (P<0.001) for women with ER+ (or ER unknown) tumors receiving 5 years of tamoxifen treatment. The benefits of tamoxifen are largely irrespective of age, menopausal status, nodal status, and use of adjuvant chemotherapy. Additionally, for women with ER+ (or ER unknown) tumors, 5 years of treatment with tamoxifen results in a relative reduction in contralateral breast cancer of 39% during 15 years of observation (Come, S. E. (2006) JAMA, 295:1434-42).

### Aromatase Inhibitors

Tamoxifen is a competitive inhibitor of estrogen at the ER. Its effects are complex and depend on the local tissue levels of co-regulatory proteins. In certain environments, tamoxifen has partial agonist effects on the ER; that is, it acts like an estrogen. In contrast, aromatase inhibitors inhibit or inactivate the enzyme responsible for the conversion of androgen substrates to estrogen. Thus, ligand for the ER is depleted; these agents have no potential agonist (estrogenic) effect. Three third-generation aromatase inhibitors are currently available. The nonsteroidal compounds anastrozole and letrozole are reversible inhibitors of aromatase, while the steroidal agent exemestane is an irreversible inactivator of the enzyme. It is not yet clear whether the pharmacological differences between these compounds or potential differences in action at the tissue level are clinically significant. Each aromatase inhibitor lowers plasma estrogen levels by approximately 98% in postmenopausal women. The effects of these agents in women with intact ovarian function are not well studied, and at this time they are indicated only in the treatment of postmenopausal women (Come, S. E. (2006) JAMA, 295:1434-42).

Today, most women with hormone receptor-positive stage I breast cancer receive primary therapy and adjuvant endocrine therapy. For the subset of women with grade 1 tumors less than 1 cm, no adjuvant systemic therapy is an appropriate option. In such patients, the 10-year mortality rate from breast cancer without adjuvant systemic therapy is 1%, and the absolute reduction in mortality attributable to the use of endocrine therapy is less than 0.5%. Conversely, the addition of adjuvant chemotherapy to endocrine therapy in hormone receptor-positive stage I cancer is generally restricted to women with high-risk features such as tumors larger than 1 cm, high tumor grade or proliferative index, weak expression of hormone receptors, overexpression or amplification of Her-2/neu, or very young age (<35 years). In this instance, the 10-year mortality rate from breast cancer is approximately 20% and remains at 13% despite the use of adjuvant endocrine therapy. The addition of chemotherapy results in a further absolute mortality reduction of 5%. For women older than 50 years, chemotherapy is less effective. Further, women with hormone receptor-positive breast cancer receiving endocrine therapy appear to derive less benefit from chemotherapy than their hormone receptor-negative counterparts (Come, S. E. (2006) JAMA, 295:1434-42).

### Aromatase Inhibitors Versus Tamoxifen

More than 40,000 women have been enrolled in trials evaluating aromatase inhibitors in early breast cancer, and several studies using varying designs have reported improvements in disease-free survival when these agents are compared with tamoxifen. In each trial, reported follow-up is measured from the onset of the randomized therapy. The Arimidex, Tamoxifen, Alone or in Combination (ATAC) trial was the first study to report results, is the largest to date, and provides the longest follow-up. A total of 9,366 women were randomized to receive 5 years of treatment with anastrozole, tamoxifen, or both. After a median follow-up of 68 months, anastrozole significantly prolonged disease-free survival (575 events with anastrozole vs 651 with tamoxifen, hazard ratio 0.87, 95% CI 0.78-0.97, p=0.01) and time-to-recurrence (402 vs 498, 0.79, 0.70-0.90, p=0.0005), and significantly reduced distant metastases (324 vs 375, 0.86, 0.74-0.99, p=0.04) and contralateral breast cancers (35 vs 59, 42% reduction, 12-62, p=0.01). Almost all patients completed their scheduled treatment, and fewer withdrawals occurred with anastrozole than with tamoxifen. Anastrozole was also associated with fewer side-effects than tamoxifen, especially gynecological problems and vascular events, but arthralgia and fractures were increased (Howell et al. (2005) Lancet, 365:60-2).

The Breast International Group (BIG) trial enrolled 8,028 women. In postmenopausal women with endocrine-responsive breast cancer, adjuvant treatment with letrozole, as compared with tamoxifen, reduced the risk of recurrent disease, especially at distant sites (Thurlimann et al. (2005) N. Engl. J. Med., 353:2747-57).

### Sequential Use of Tamoxifen and Aromatase Inhibitors

The Intergroup Exemestane Study (IES) randomized 4,742 women who had taken 2 to 3 years of tamoxifen therapy to either continue tamoxifen for a total of 5 years or switch to exemestane to complete a total of 5 years of adjuvant endocrine therapy. For the primary end point of disease-free survival, the sequence was superior to continued tamoxifen, with an absolute difference of 4.7% (91.5% vs 86.8%; HR, 0.68 [95% CI, 0.56-0.82]; P<0.001) and a median follow-up of 30.6 months. At a median follow-up of 37.4 months with 339 deaths recorded, there was a insignificant trend in overall survival favoring sequential therapy (Come, S. E. (2006) JAMA, 295:1434-42; Jones, S. E. (2006) Clin. Breast Cancer, 6 Suppl 2:S41-S44).

### Adverse Effects

The safety and tolerability of tamoxifen have been well defined during the past 2 decades; a large amount of data from both therapeutic and prevention trials (compared to placebo) is available. The experience with aromatase inhibitors is more limited; the longest follow-up available in the adjuvant setting is supplied by the ATAC trial, with a median follow-up of 68 months. In this trial, 92% of patients completed the 5 years of study medication. However, the other major trials have reported adverse effects with median follow-ups of only 2 to 3 years thus far. In the randomized adjuvant trials, aromatase inhibitors are tolerated at least as well as tamoxifen. In the ATAC trial, the withdrawal rate for drug-related adverse events was 5.1% for anastrozole and 7.2% for tamoxifen. Menopausal symptoms manifest as hot flashes, night sweats, and mood disturbance are common with both tamoxifen and aromatase inhibitors. The aromatase inhibitor produced fewer hot flashes than tamoxifen in the ATAC trial (P<0.001) and in the BIG 1-98 trial, but this difference was not observed in trials in which women were randomized to continue taking tamoxifen for 5 years vs switch to an aromatase inhibitor after the initial 2 years of tamoxifen. Loss of libido occurred at similar frequency with exemestane and tamoxifen (25% to 28%) in the IES trial and appeared to worsen, at least over the first 24 months of follow-up. During the first 24 months of the ATAC trial, loss of sexual interest was more common in women taking anastrozole than tamoxifen (16% vs 9%, P=0.05). Vaginal discharge and bleeding were more common in women taking tamoxifen in each of the trials, while joint and muscle pain were more common in women receiving aromatase inhibitors. Nausea and other gastrointestinal adverse effects have been reported in equal frequency for women taking either aromatase inhibitors or tamoxifen, although an excess of diarrhea has been reported for exemestane vs tamoxifen (4.3% vs 2.3%, P<0.001).

The most serious complications of tamoxifen are related to its estrogen agonist effects. In an overview of available trials, 5 years of tamoxifen therapy was associated with a risk ratio of 4.2 for endometrial cancer, raising the 10-year risk per 1000 from 3 in the control population to 11 with tamoxifen (P<0.001). One to 2 years of tamoxifen treatment raised the relative risk to approximately 2.0. Nevertheless, the increase in endometrial cancer with 5 years of tamoxifen was only half as large as the decrease in contralateral breast cancer. Further, the 10-year risk of mortality from endometrial cancer in tamoxifen users was only 2 per 1000. The NSABP tamoxifen prevention trial reported a risk ratio of 4.01 (95% CI, 1.70-10.90) for tamoxifen use in women older than 50 years; the cumulative incidence of endometrial cancer at 66 months of follow-up was 5.4 per 1000 women for the placebo group vs 13.0 per 1000 women in the tamoxifen group (Fisher et al. (1998) J. Natl. Cancer Inst., 90:1371-88).

The major concerns regarding the safety of aromatase inhibitors are a consequence of the marked decrease in estrogen levels. After 5 years of follow-up in the ATAC trial, the relative risk of fractures was 1.49 (95% CI, 1.25-1.77; P<0.001) for women taking anastrozole with a fracture rate of 11% vs 7.7% for women taking tamoxifen.

Indeed, bone loss is a troubling side effect of aromatase inhibitors. Current evidence suggests a minor bone loss during treatment with the steroidal aromatase inhibitor exemestane compared to placebo and a non-significant increase in fracture rate during treatment with exemestane when compared to tamoxifen. Such a difference could be due to the bone-sparing effects of tamoxifen. For the nonsteroidal inhibitors letrozole and anastrozole, there is a non-significant increase in fracture rate for letrozole compared to placebo. In contrast, both anastrozole and letrozole were found to significantly increase fracture rate compared to tamoxifen when administered as monotherapy or given sequentially. While an increased fracture rate has detrimental effects, evidence suggests that enhanced bone loss may be preventable through careful bone mineral density (BMD) assessment and treatment with bisphosphonates (Lonning, P. E. (2006) Int. J. Gynecol. Cancer, 16 Suppl 2:518-20; Gasser et al. (2006) Bone, 39:787-95). But bisphosphonates have a distressing, untreatable side effect: osteonecrosis of the mandible (Migliorati et al. (2005) Cancer, 104:83-93). Patients who develop bisphosphonate-associated jaw osteonecrosis present with such symptoms as sensations of heaviness or numbness in the jaw, pain, swelling, infection, loose teeth, and exposed bone which often never heals.

A second side effect of aromatase inhibitors is hyperlipidemia and cardiovascular disease. For example, a total of 43.6 percent of patients in the letrozole group and 19.2 percent of patients in the tamoxifen group had hypercholesterolemia reported at least once during treatment. More women in the letrozole group had grade 3, 4, or 5 cardiac events (2.1 percent vs. 1.1 percent, P<0.001) (Thurlimann et al. (2005) N. Engl. J. Med., 353:2747-57).

Reduction in fracture incidence and reduction of cardiovascular disease could be achieved by combining statin therapy with aromatase therapy.

### Cholesterol Lowering and Statins

Most circulating cholesterol is manufactured internally, typically about 1000 mg/24 hours, out of the carbohydrate metabolism, by the HMG-CoA reductase pathway. Cholesterol, both from dietary intake and secreted into the duodenum as bile from the liver, is typically absorbed at a rate of 50% by the small intestines. The typical diet in the United States and many other Western countries is estimated as adding about 200-300 mg/day to intestinal intake, much smaller than that secreted into the intestine in the bile. Thus internal production is an important factor.

Cholesterol is not water-soluble and is therefore carried in the blood in the form of lipoproteins, the type being determined by the apoprotein, a protein coating that acts as an emulsifier. The relative balance between these lipoproteins is determined by various factors, including genetics, diet, and insulin resistance. Low density lipoprotein (LDL) and very low density lipoprotein (VLDL) carry cholesterol towards tissues, and elevated levels of these lipoproteins are associated with atheroma formation (fat-containing deposits in the arterial wall) and cardiovascular disease. High density lipoprotein, in contrast, carries cholesterol back to the liver and is associated with protection against cardiovascular disease.

Statins act by competitively inhibiting HMG-CoA reductase, the first committed enzyme of the HMG-CoA reductase pathway. By reducing intracellular cholesterol levels, they cause liver cells to upregulate expression of the LDL receptor, leading to increased clearance of low-density lipoprotein from the bloodstream.

Direct evidence of the action of statin-based cholesterol lowering on atherosclerosis was presented in the ASTEROID trial, which demonstrated regression of atheroma employing intravascular ultrasound (Nissen et al. (2006) JAMA, 295:1556-65).

### Non-Cholesterol Related Statin Actions

Statins exhibit actions beyond lipid-lowering activity in the prevention of atherosclerosis. Researchers believe that statins prevent cardiovascular disease via four proposed mechanisms (all subjects of a large body of biomedical research): improving endothelial function, modulating inflammatory responses, maintaining plaque stability, and preventing thrombus formation (Furberg, C. D. (1999) Circulation, 99:185-8).

### Indications and Uses of Statins

Statins, the most potent cholesterol-lowering agents, lower LDL-cholesterol (so-called “bad cholesterol”) by 30-50%. However, they have less effect than the fibrates or niacin in reducing triglycerides and raising HDL-cholesterol (“good cholesterol”). Professional guidelines generally require that the patient has tried a cholesterol-lowering diet before statin use is considered. In practice, however, a diet-induced cholesterol reduction of more than 10% is unusual, and many patients do not achieve their targets through dietary approaches.

The indications for the prescription of statins have broadened over the years. Initial studies, such as the Scandinavian Simvastatin Survival Study (4S), supported the use of statins in secondary prevention for cardiovascular disease, or as primary prevention only when the risk for cardiovascular disease was significantly raised (Wilson et al. (1998) Circulation, 97:1837-47). Indications were broadened considerably by studies such as the heart protection study (HPS), which showed preventative effects of statin use in specific risk groups, such as diabetics (Collins et al. (2003) Lancet, 361:2005-16). The ASTEROID trial published in 2006, using only a statin at high dose, and achieving lower than usual target calculated LDL values, showed disease regression within the heart arteries using IVUS (Nissen et al. (2006) JAMA, 295:1556-65).

Based on clinical trials, the National Cholesterol Education Program guidelines, and the increasing focus on aggressively lowering LDL-cholesterol, the statins continue to play an important, indeed dominant and increasing role in both the primary and secondary prevention coronary heart disease, myocardial infarction, stroke and peripheral artery disease.

Research continues into other areas where statins also appear to have a favorable effect: inflammation, dementia, neoplastic conditions, pulmonary hypertension, and prevention of fracture.

### Statins Prevent Fracture

Although there is still some controversy, statins appear to reduce the incidence of fracture (see, e.g., Rejnmark et al. (Osteoporos. Int. (2004) 15:452-458). In one study of 91,052 patients, 28,063 were prescribed statins and 2195 were prescribed nonstatin lipid-lowering medications. In the adjusted analyses, statin use was associated with a 36% (odds ratio, 0.64; 95% confidence interval, 0.58-0.72) reduction in fracture risk when compared with no lipid-lowering therapy and a 32% (odds ratio, 0.67; 95% confidence interval, 0.50-0.91) reduction when compared with nonstatin lipid-lowering therapy (Scranton et al. (2005) Arch. Intern. Med., 165:2007-12).

In a case-control study, 124,655 fracture cases were compared with 373,962 age and gender-matched controls (Rejnmark et al. (2006) Calcif. Tissue Int., 79:27-36). Use of statins was associated with a reduced risk of any fracture (adj. OR 0.87; 95% CI, 0.83-0.92) and hip fractures (adj. OR 0.57; 95% CI, 0.48-0.69). Risk of hip fracture decreased with increased accumulated dose of statins. This was true in men and in women and in subjects younger and older than 65 years of age. However, fracture risk was not reduced in patients treated with pravastatin (adj. OR 1.02; 95% CI, 0.89-1.17) or non-statin lipid lowering drugs (adj. OR 0.99; 95% CI, 0.86-1.15). Moreover, the reduced fracture risk in users of lipid lowering drugs is apparently specifically related to users of non-pravastatin statins and was not due to the “healthy drug user effect” (Ray, W. A. (2003) Am. J. Epidemiol., 158:915-20) as an explanation for the reduced fracture risk in users of statins.

Furthermore, in both symptomatic and non-symptomatic vertebral fractures in the elderly (N=3469), long-term statin use is significantly associated with a 50% lower vertebral fracture risk (Schoofs et al. (2004) J. Bone Miner Res., 19:1525-30).

### Statin Combination Drugs

Notably, statins have proven effective when combined with other medications for different indications. For example, VYTORIN® (Merck) is a combination of simvastatin (ZOCOR®) and ezetimibe (ZETIA) for treating hyperlipidemia; CADUET® (Pfizer) is a combination of amlodipine besylate (NORVASC®) and atorvastain (LIPITOR®) for treating hypertension and hypercholesterolemia; and Torcetrapib/Atorvastatin (Pfizer) is a combination of torcetrapib and atorvastain (LIPITOR®) for raising high density lipoprotein (HDL) and reducing LDL (low density lipoprotein) cholesterol.

### Definitions

The following definitions are provided to facilitate an understanding of the present invention.

“Pharmaceutically acceptable” refers to entities and compositions that are physiologically tolerable and do not typically produce an allergic or similar untoward reaction when administered to an animal, particularly a human. Pharmaceutically acceptable carriers are preferably approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, solubilizer, emulsifier, auxiliary agent or vehicle with which an active agent of the present invention is administered. Such pharmaceutically acceptable carriers can be sterile liquids, such as water (may be deionized), alcohol (e.g., ethanol, isopropanol), oils (including those of petroleum, animal, vegetable or synthetic origin, such as peanut oil, soybean oil, mineral oil, sesame oil and the like), and other organic compounds or coploymers. Water or aqueous saline solutions and aqueous dextrose and glycerol solutions may also be employed as carriers. Carriers may also be diluents of various buffer content (e.g., Tris-HCl, acetate, phosphate), pH and ionic strength. Detergents and solubilizing agents (e.g., Tween 80, Polysorbate 80), anti-oxidants (e.g., ascorbic acid, sodium metabisulfite), preservatives (e.g., Thimerosal, benzyl alcohol) and bulking substances (e.g., lactose, mannitol) may also be used. The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. Suitable pharmaceutical carriers and other agents of the compositions of the instant invention are described in “Remington's Pharmaceutical Sciences” by E. W. Martin (Mack Pub. Co., Easton, Pa.) and “Remington: The Science And Practice Of Pharmacy” by Alfonso R. Gennaro (Lippincott Williams & Wilkins, 2005).

The term “chemotherapeutic agent” refers generally to any compound that exhibits anticancer activity. Chemotherapeutic agents include, but are not limited to: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). Preferably, the chemotheraputic agent is selected from the group consisting of: paclitaxel (Taxol®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

A “therapeutically effective amount” of a compound or a pharmaceutical composition refers to an amount effective to prevent, inhibit, or treat the symptoms of a particular disorder or disease. For example, “therapeutically effective amount” may refer to an amount sufficient to modulate tumor growth or metastasis in an animal, especially a human, including without limitation decreasing tumor growth or size or preventing formation of tumor growth in an animal lacking any tumor formation prior to administration, i.e., prophylactic administration. The term may also refer to an amount sufficient to prevent, slow, or halt progression of osteoporosis or other bone disorders.

“Concurrently” means (1) simultaneously in time, or (2) at different times during the course of a common treatment schedule.

“Sequentially” refers to the administration of one component of the method followed by administration of the other component. After administration of one component, the next component can be administered substantially immediately after the first component, or the next component can be administered after an effective time period after the first component; the effective time period is the amount of time given for realization of maximum benefit from the administration of the first component.

An “estrogen related cancer” refers to cancers that are modulated by estrogen. Estrogen related cancers include, without limitation, breast cancer and ovarian cancer. Typically, the estrogen related cancers are estrogen receptor positive.

### Pharmaceutical Compositions and Uses Thereof

The pharmaceutical compositions of the instant invention comprise at least one aromatase inhibitor and at least one HMG-CoA reductase inhibitor (statin). Pharmaceutically acceptable salts of the aromatase inhibitors and HMG-CoA reductase inhibitor are also encompassed. In a preferred embodiment, the pharmaceutical compositions further comprise at least one pharmaceutically acceptable carrier. In yet another embodiment, the pharmaceutical compositions may further comprise at least one chemotherapeutic agent.

Aromatase inhibitors include, but are not limited to anastrozole, letrozole, or exemestane. HMG-CoA reductase inhibitors (statins) include, but are not limited to, atorvastatin (e.g., LIPITOR®), fluvastatin (e.g., LESCOL®, CANEF®), lovastatin (e.g., MEVACOR®), mevastatin, pitavastatin, pravastatin (e.g., PRAVACHOL® or SELEKTINE®), rosuvastatin (e.g., CRESTOR®), and simvastatin (e.g., ZOCOR®). In a particular embodiment, the pharmaceutical composition comprises anastrozole and simvastatin.

The pharmaceutical compositions of the present invention can be administered by any suitable route, for example, by injection, orally, pulmonary, nasally, controlled release devices, or other modes of administration.

In a preferred embodiment, the at least one aromatase inhibitor and at least one HMG-CoA reductase inhibitor are contained within the same pharmaceutical composition. In a particular embodiment, the pharmaceutical composition is in the form of a pill, tablet, or capsule.

At least one aromatase inhibitor and at least one HMG-CoA reductase inhibitor may also be present in separate pharmaceutical compositions. The pharmaceutical composition comprising at least one HMG-CoA reductase inhibitor may be administered to a patient concurrently or sequentially with the pharmaceutical composition comprising at least one aromatase inhibitor. For example, an aromatase inhibitor can be administered to a patient first, and then followed by administration of the at least one HMG-CoA reductase inhibitor, as needed (e.g., by determining/monitoring the bone health of the patient). In a particular embodiment, the bone health of the patient and/or status of the cancer is monitored during and/or after the administration of the pharmaceutical compositions.

The pharmaceutical compositions may be used to treat cancer in a patient in need thereof. Particularly, the compositions may be administered to a patient to treat estrogen related cancers such as breast cancer.

The following example describes illustrative methods of practicing the instant invention and is not intended to limit the scope of the invention in any way.

### EXAMPLE

DIAGNOSIS. Breast cancer would be diagnosed by biopsy. The biopsy could be excisional, ideally with removal of the entire tumor. Alternatively, a needle biopsy could be performed. Pathologic examination of the excised tissue may then be performed.

DOSAGE. Upon diagnosing the patient with cancer, composition of the instant invention may be administered. Ideally, the composition will be a pill or capsule and contain, for example, 1 mg anastrozole/20 mg simvastatin to be administered daily, 2.5 mg letrozole/20 mg simvastatin to be administered daily, or 2.5 mg exemestane/20 mg simvastatin to be administered daily.

FOLLOW-UP. The proper dose of aromatase inhibitor may vary by patient. Those of skill in the art could determine the proper dose by aiming for complete suppression of estrone (E1), estradiol (E2), and estrone sulphate (E1S) in serum after the seventh day of treatment. The doses of anastrozole, letrozole, and exemestane listed above are generally adequate for this purpose.

The proper dose of simvastatin may also vary by patient. The proper dose could be determined by one of skill in the art by measuring total serum cholesterol before therapy and after one month of therapy. Ideally the statin should reduce total serum cholesterol below 200 mg/dl in order to prevent the vascular disease induced by aromatase inhibitors and risk of fracture associated with aromatase inhibitors.

Bone mineral density (BMD) may be measured before the onset of therapy and every three months thereafter to assess bone health. This measurement is now generally done before prescribing an aromatase inhibitor. If BMD began to decline, the dose of simvastatin could be increased to 40 mg per day from 20 mg per day, to a maximum dose of 80 mg per day.

The statin doses described above refer to simvastatin. Atorvastatin is approximately twice as potent as simvastatin and so a lower dose would be required. Similarly, rosuvastatin is more potent than atorvastatin and would therefore require an even lower dose.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A pharmaceutical composition comprising at least one aromatase inhibitor, at least one HMG-CoA reductase inhibitor, and at least one pharmaceutically acceptable carrier.

2. The pharmaceutical composition of claim 1, further comprising at least one additional chemotherapeutic agent.

3. The pharmaceutical composition of claim 1, wherein said aromatase inhibitor is selected from the group consisting of anastrozole, letrozole, and exemestane.

4. The pharmaceutical composition of claim 1, wherein said HMG-CoA reductase inhibitor is selected from the group consisting of atorvastatin, fluvastatin, lovastatin, mevastatin, pitavastatin, pravastatin, rosuvastatin, and simvastatin.

5. The pharmaceutical composition of claim 1, wherein said aromatase inhibitor is anastrozole and said HMG-CoA reductase inhibitor is simvastatin.

6. A method of treating cancer comprising the administration to a patient in need thereof at least one aromatase inhibitor and at least one HMG-CoA reductase inhibitor.

7. The method of claim 6, wherein said aromatase inhibitor and said HMG-CoA reductase inhibitor are administered concurrently.

8. The method of claim 6, wherein said aromatase inhibitor and said HMG-CoA reductase inhibitor are administered sequentially.

9. The method of claim 7, wherein said aromatase inhibitor and said HMG-CoA reductase inhibitor are contained within the same pharmaceutical composition.

10. The method of claim 6 further comprising the administration of an additional chemotherapeutic agent.

11. The method of claim 8, wherein said HMG-CoA reductase inhibitor is administered after assessment of the patient's bone health.

12. The method of claim 6, wherein said cancer is an estrogen related cancer.

13. The method of claim 12, wherein said cancer is breast cancer.

14. The method of claim 6, wherein said HMG-CoA reductase inhibitor is administered to reduce the bone loss caused by said aromatase inhibitor.
